(19) United States
(12) Patent Application Publication
(10) Pub. No.: US 2006/0195111 A1
(43) Pub. Date: Aug. 31, 2006

(54) Universal positioning block assembly

(75) Inventor: Pierre Couture, Montreal (CA)

Correspondence Address: OGILVY RENAULT LLP, 1981 MCGILL COLLEGE AVENUE, SUITE 1600, MONTREAL, QC, H3A2Y3, CA

(73) Assignee: ORTHOSOFT INC.

(21) Appl. No.: 11/042,264

(22) Filed: Jan. 25, 2005

(51) Int. Cl. A61F 5/00

(52) U.S. Cl. 606/86

(57) ABSTRACT

A universal positioning block for use in bone surgery, permitting up to six degrees-of-freedom movement relative to a bone element to which it is fixed. The positioning block comprises a main block element that is releasably engageable to the bone element such that the main block element is rotatable in a flexion-extension rotation plane and translatable along a medio-lateral axis relative to the bone element. A slider element is engaged with the main block element such that it is translatable along an antero-posterior axis and rotatable in a varus-valgus rotation plane relative thereto and a holder element is engaged with the slider element such that it is translatable relative thereto along a proximal-distal axis and may also further be rotatable in a medio-lateral rotation plane, the holder element being habilitated for engaging a surgical tool guide element.

### TECHNICAL FIELD

The present invention relates generally to a surgical tool for use in bone surgery, and particularly to a multiple degree-of-freedom positioning block assembly for joint replacement surgery. More specifically, the present invention is directed to a universal positioning block assembly for use with a computer assisted surgery (CAS) system in total knee replacement surgery. More specifically, the present invention provides for a positioning block assembly which when fixed to a bone element of a joint, will assist a surgeon by providing the location of a surgical tool guide block, which surgical tool guide block is used by the surgeon so as to perform the cutting of the bone element.

### BACKGROUND

Although emphasis is put on total joint replacement surgery, it is to be understood that the following invention may also apply to other related bone surgeries such as for example, unicondylar osteotomy, unicompartmental joint replacement, etc.

Total joint replacement surgery requires the removal of the joint structure and the replacement thereof with an artificial joint. Generally speaking, each of the two opposite bones leading into the joint structure need to be completely cut at a point removed from the joint so as b allow the removal of the joint. In order for such a surgery to be as successful as possible, and to minimize any post-operative difficulties, a surgeon needs to severe the bone as accurately as possible, taking into account the normal variation in body physiology from one patient to the next.

Accuracy of cuts and drilled holes is important in joint replacement surgery such as in knee arthroplasty, wherein installation of the implants such that the kinematics of the natural knee are duplicated as much as possible, is important to the success of the total knee replacement. To achieve this, the use of CAS systems for orthopedic operations in general, and for total knee replacement surgery in particular, is becoming increasingly more commonplace with advancements in CAS equipment that ensure improved accuracy, near fail safe operation and increased ease of use.

Known optical, radio frequency and magnetic based CAS systems employ passive and active trackable elements affixed to objects, such as surgical tools and patient bone references, in order to permit the determination of position and orientation of the objects in three-dimensional space. Preoperatively taken images, computer generated models created from preoperative patient scans or intra operative landmark digitization are used to provide accurate patient anatomical information to which the real-time position of the same anatomical elements can be registered or calibrated, thereby permitting subsequent tracking of the anatomical elements and display of these elements relative to the surgical tools used during the surgery.

Total knee replacement surgery, for example, may require several precise cuts to be made in the femur and tibia to completely remove the knee joint, such that the implant may fit correctly and best replicates the geometry of a natural healthy knee. Alternatively a single cut to the femur and the tibia may do. To perform these steps, in both conventional and CAS total knee replacement, it is well known to use a tool or implement known as a surgical tool guide block which provides a drill and/or cutting guide to assist the surgeon to perform the steps required to prepare the femur and tibia for receiving the implant. Thus in traditional, known CAS surgery, the surgical tool guide block would be drilled to that part of the bone to be severed, while in other bone CAS systems, the surgical tool guide block would also be screwed into that part of the bone to be severed, and its position would be determined through known computer assisted surgery.

Present CAS systems using tracked CAS positioning blocks permit improved visualization of the surgical tool guide block relative to the bone elements of the femur, requiring fewer fixed anatomical reference surfaces. However, to best permit the fixation of the surgical tool guide block in a determined position, presently a surgeon must use a positioning block which requires controllable adjustment of several degrees of freedom. While certain flexibility is provided by total knee replacement positioning blocks of the prior art, there nevertheless remains a need for a positioning block permitting additional controllable flexibility of movement, and being adapted for use with a CAS system. One such system is described in ORTHOsoft Inc. U.S. patent application Ser. No. 10/357,493 entitled “UNIVERSAL POSITIONING BLOCK”, by Couture et al. Once the positioning block has been positioned so as to have its reference surface located in a position corresponding to the predetermined emplacement of the bone cutting guide, locating pegs are secured to the bone so that the bone cutting guide may be properly placed once the positioning block is removed. However, this two step process may lead to some imprecision in the final cutting plane. Furthermore, the positioning block being translatable along a proximal-distal axis corresponding to the longitudinal axis of a polyaxial screw positioned at the distal end of the bone, the proximal translation is limited by the distance of the head of the polyaxial screw with regards to the distal end of the bone.

### SUMMARY

Accordingly, it is an object of the present invention to provide a positioning block assembly for bone surgery allowing improved precision in the positioning of a surgical tool guide block.

There is therefore provided, in accordance with the one aspect of the present invention, a positioning block assembly for use in bone surgery, to be releasably affixed to a bone element to undergo surgery, the position block assembly permitting up to six degrees-of-freedom movement relative to the bone element to which it is to be releasably affixed. The positioning block assembly comprises: a main block element being removably attachable to the bone element such that the main block element is rotatable in a flexion-extension rotation plane and translatable along a medio-lateral axis relative to the bone element; a slider element being slidably engaged with the main block element such that it is translatable along an antero-posterior axis and rotatable in a varus-valgus rotation plane relative to the block element and a holder element being slidably engaged with the slider element such that it is translatable relative to the slider element along a proximal-distal axis and rotatable in a medio-lateral rotation plane, the holder element being configured and disposed to releasably engage a surgical tool guide element.

In accordance with another aspect of the present invention, there is provided a computer assisted bone surgery system comprising: a positioning block assembly being releasably affixed to a bone element; means for determining the position and orientation of the positioning block relative to the bone element; a guide element being releasably engaged the positioning block assembly; means for identifying a desired position of the positioning block assembly relative to the bone element, such that the guide element is located in a selected position relative to the bone element, such that a cut can be made in the bone element at the selected position and a display capable of indicating when the desired location of the positioning block assembly is reached.

In accordance with a further aspect of the present invention, there is provided a method of installing a guide element on a bone element, the guide element being releasably engaged to a positioning block assembly, the method comprising: fastening the positioning block assembly to the bone element; determining a desired position of the guide element engaged to the positioning block assembly relative to the bone element; adjusting at least one of the position and orientation of the positioning block assembly until the guide element is in the desired position and securing the guide element on the bone element at the predetermined location.

### BRIEF DESCRIPTION OF THE FIGURES

Embodiments of the invention will now be described by way of examples only with reference to the accompanying drawings, in which:

FIG. 1 is a perspective view of a universal positioning block assembly operatively engaged with a surgical tool guide block, mounted to a femur.

FIG. 2 is a top view of the universal positioning block assembly of FIG. 1.

FIG. 3 is a perspective view of a bone anchor mounted to a femur.

FIG. 4 is a perspective view of an alternate embodiment of the bone anchor mounted to a femur.

FIG. 5 is a perspective view of a bone anchor assembled with a main block and an antero-posterior slider, mounted to a femur.

FIG. 6 is a perspective view of an alternative embodiment of FIG. 5.

FIG. 7 is a cross-sectional view of the main block.

FIG. 8 is a cross-sectional view of an alternate embodiment of the main block.

FIG. 9 is a perspective view of a bone anchor assembled with a main block, an antero-posterior slider, a guide block holder and a tracker member, mounted to a femur.

FIG. 10 is a perspective view of the guide block holder.

FIG. 11 is a bottom view of the guide block holder of FIG. 10.

FIG. 12 is a perspective view of an alternate embodiment of a guide block holder.

FIG. 13 is a bottom view of the guide block holder of FIG. 12.

FIG. 14 is a schematic flow chart of the method used to install the universal positioning block of the present invention to a bone element.

### DETAILED DESCRIPTION

Throughout this application, a particular embodiment of the present invention will be referred to as a universal positioning block assembly or simply positioning block assembly, and is for use in bone surgery, such as for example, total or partial joint replacement surgery of the knee, elbow, hip, shoulder or other joint, unicondylar osteotomy, unicompartmental knee replacement, total knee arthroplasty, high tibial osteotomy, etc. Furthermore, the positioning block assembly may be used as in conjunction with a computer assisted surgical (CAS) system or may be used on its own. The positioning block assembly comprises a guide block holder that is releasabily, operatively engageable with a surgical tool guide block and is adapted to accurately position and align the surgical tool guide block. The surgical tool guide block is adapted for guiding a surgical tool and it is to be understood that such a surgical tool as defined herein includes all surgical instruments necessary for bone surgery and joint replacement surgery, for example those which can remove bone from a bone element, such as drills, rasps and saws and that such a surgical tool guide block is similarly adapted for any surgical instrument necessary for joint replacement surgery, for example those which can remove bone from a bone element. It may be further understood that the surgical tool guide block may also be a surgical device.

The universal positioning block assembly may be trackable by a computer assisted surgical (CAS) system which provides means for determining the position, orientation and movement of the positioning block assembly in a three dimensional space, and permits the positioning block assembly to be visualized, for example using a display, relative to the patient anatomy. The CAS system further provides means for determining a desired position of the positioning block assembly relative to a bone element, whether from a real patient, a cadaver or a model. The CAS system further provides means for indicating where to fasten the surgical tool guide block on such a bone element such that it can be affixed into the desired position. Additionally, the present positioning block assembly may be used with both CT-based and image-less CAS systems or fluoroscopic systems. The CAS system may, in other words, use either computer generated anatomical models created from pre-operatively taken scans, such as CT scans, or use intra-operatively generated bone surface models created by digitizing a plurality of points and anatomic landmarks on the surface of the bone element, to relate the position of the positioning block assembly to the bone elements of the patient.

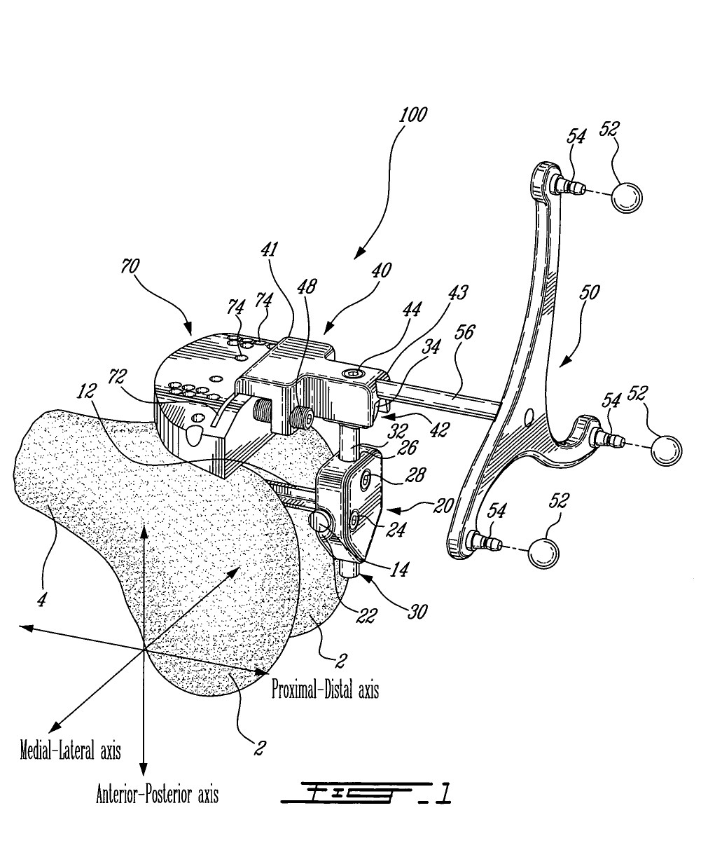

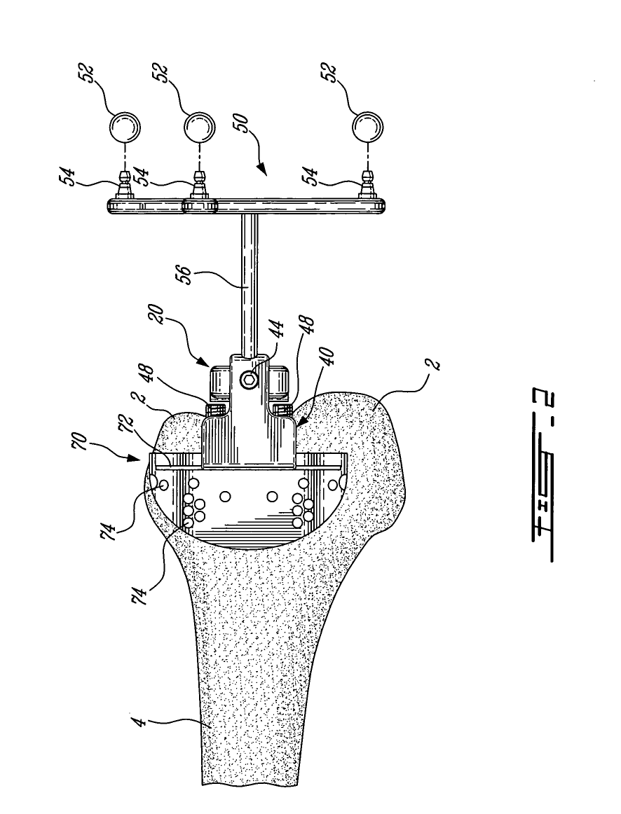

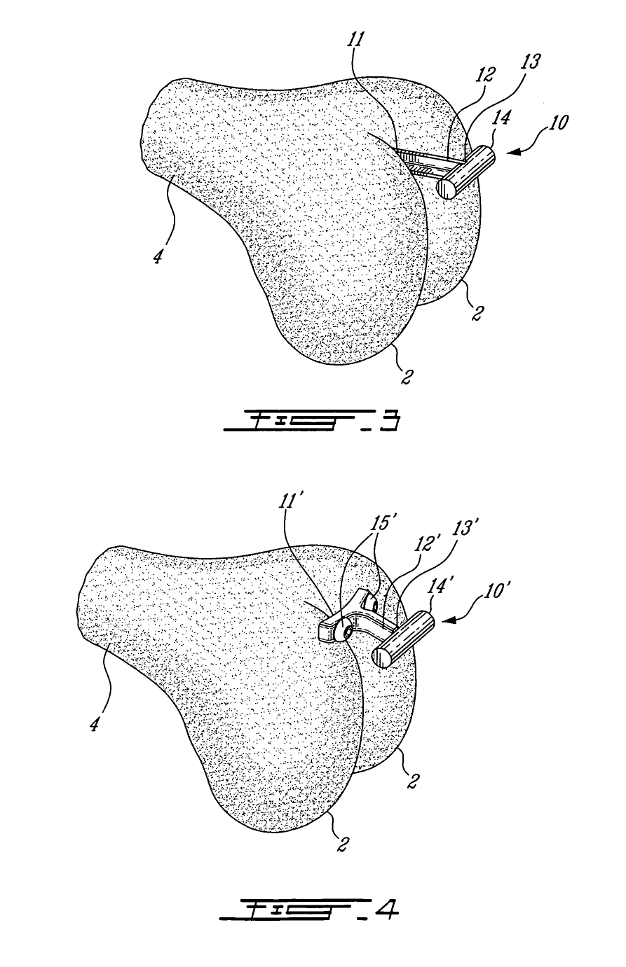

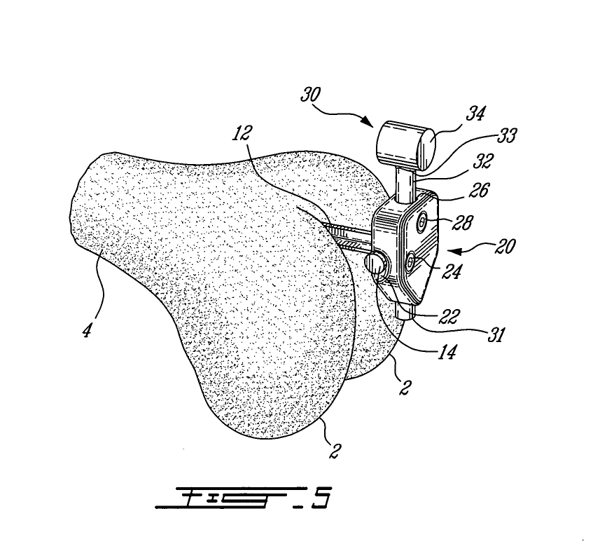

Referring to FIGS. 1 and 2, the universal positioning block assembly 100 comprises generally a main block 20, a guide block holder 40 for holding a surgical tool guide block 70 and a tracker member 50 connected to the guide block holder 40. It is to be understood that the tracker member 50 may be omitted if the positioning block assembly 100 is not to be used with a CAS system. Referring to FIG. 3, a bone anchor 10 is used to mount the universal positioning block assembly 100 to a femur 4. The bone anchor 10 may generally comprise opposed first 11 and second ends 13, a body 12 which may have a cross-shaped cross-section to prevent rotation about its longitudinal axis and a head 14, which may be generally cylindrical in shape. It is to be understood that other body 12 cross-section geometries that prevent rotation may also be used. The bone anchor 10 may be impact engaged into a distal end of the femur 4, for example, in the intercondylar notch, between the two distal condyles 2 in such a way that the head 14 has a longitudinal axis generally parallel to the medio-lateral axis and is generally perpendicular to the flexion-extension rotation plane. In an alternative embodiment, the body 12 of the bone anchor 10 may be in the shape of a screw and would be screwed into the femur 4. In an further alternative embodiment, shown in FIG. 4, a bone anchor 10′ may generally comprise opposed first 11′ and second ends 13′, a body 12′ having a generally funnel or triangular shaped body 12′ and a head 14′, which may be generally cylindrical in shape. The bone anchor 10′ is attached to a distal end of the femur 4 over the intercondylar notch, by screwing a cortical screw 15′ in each of the two distal condyles 2 in such a way that the head 14′ has a longitudinal axis generally parallel to the medio-lateral axis and is generally perpendicular to the flexion-extension rotation plane. It is further understood that additional configurations and dispositions of the bone anchor 10 may be contemplated to be within the ambit of the present invention. In particular, as long as a bone anchor 10 is provided so that once anchored into the, for example, head of the femur, a head 14 is disposed generally parallel to the medio-lateral axis and is generally perpendicular to the flexion-extension rotation plane.

Referring to FIG. 5, the main block 20 is shown as being provided with an aperture 22, for receiving the head 14 of the bone anchor 10 and an aperture 26 for receiving the body 32 of an antero-posterior slider 30 therein. The main block 20 is engaged to the bone anchor 10 by sliding the main block 20 onto the bone anchor 10 such that the head 14 of the bone anchor 10 slides within its correspondingly sized and configured main block aperture 22. Thus positioned, the main block 20 may be translated along the medio-lateral axis and rotated in the flexion-extension rotation plane. A medio-lateral translation/flexion-extension angle friction locking screw 24 engages the head 14 of the bone anchor 10 to retain a selected position relative to the bone anchor 10.

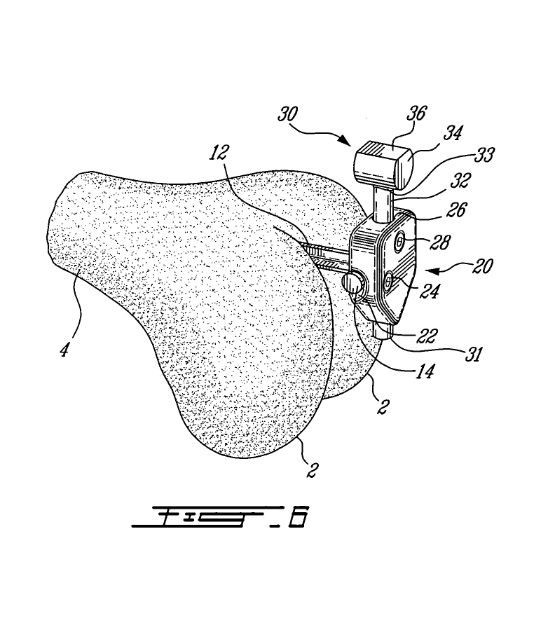

The antero-posterior slider 30 may generally comprise opposed first 31 and second ends 33, the body 32, which may be generally cylindrical in shape and has a longitudinal axis that generally corresponds to the antero-posterior axis and is generally perpendicular to the varus-valgus rotation plane, and a head 34, which may be generally cylindrical in shape. The antero-posterior slider 30 is engaged to the main block 20 by sliding the body 32 of the antero-posterior slider 30 within a correspondingly sized and configured main block aperture 26. Thus positioned, the antero-posterior slider 30 may be translated along the anterior-posterior axis and rotated in the varus-valgus plane. An antero-posterior translation/varus-valgus angle friction locking screw 28 engages the body 32 of the antero-posterior slider 30 to retain a selected position relative to the main block 20. In an alternative embodiment, shown in FIG. 6, the antero-posterior slider head 34 has a flattened top part 36 or may have a flattened lateral side.

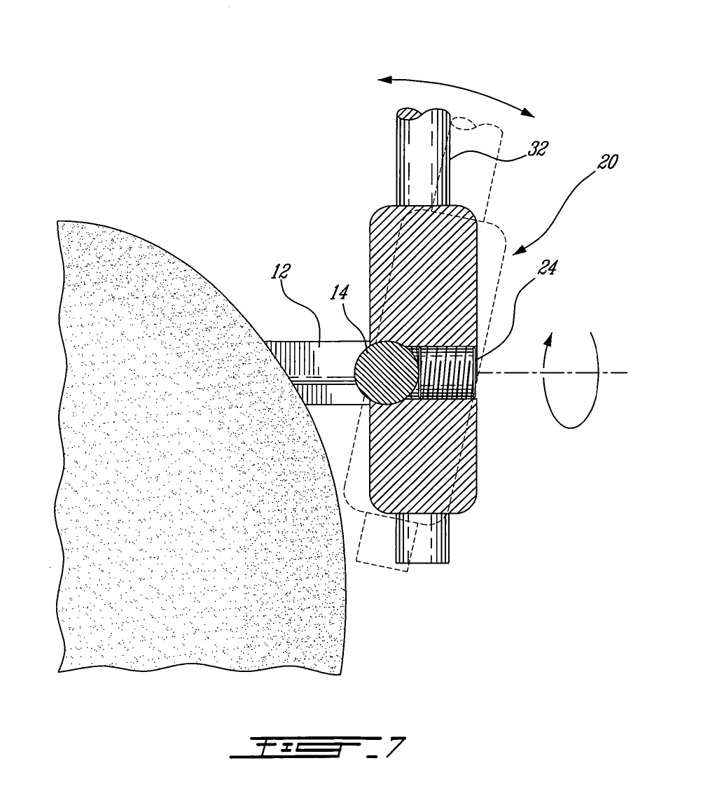

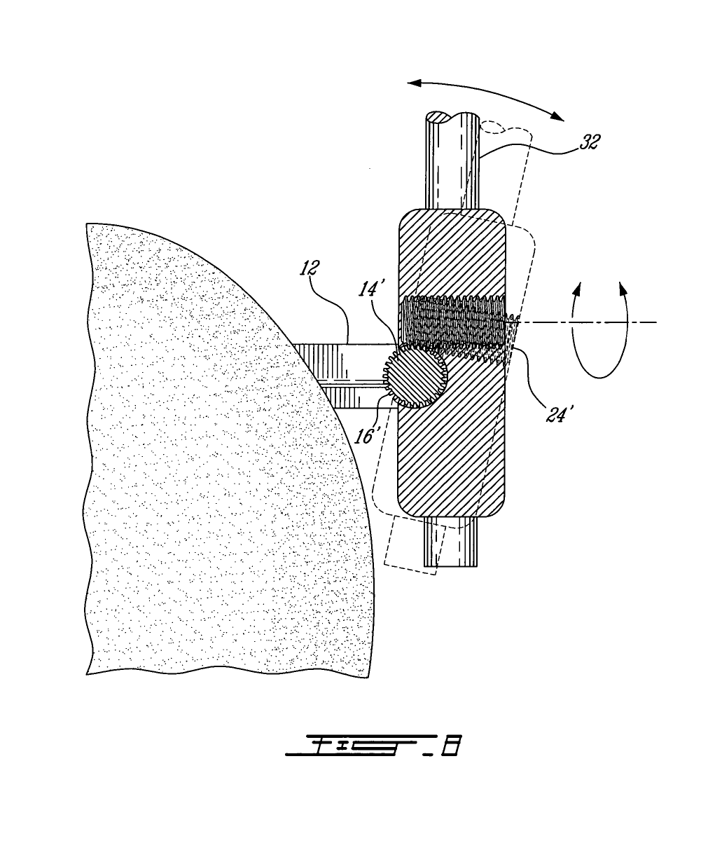

In FIG. 7, there is shown a cross-section of the main block 20 that illustrates the medio-lateral translation/flexion-extension angle friction locking screw 24 engaging the head 14 of the bone anchor 10 to retain a selected position relative to the bone anchor 10. In an alternate embodiment shown in FIG. 8, the friction locking screw 24 may be replaced by an endless screw 24′, which engages a bone anchor head 14′ having a series of indentations 16′ which are reciprocal to the threads of the medio-lateral translation/flexion-extension angle endless screw 24′. Similarly, the anterior-posterior translation/varus-valgus angle friction locking screw 28 may be replaced by an endless screw, which engages a antero-posterior sliderbody having a series of indentations which are reciprocal to the threads of the anterior-posterior translation/varus-valgus angle endless screw.

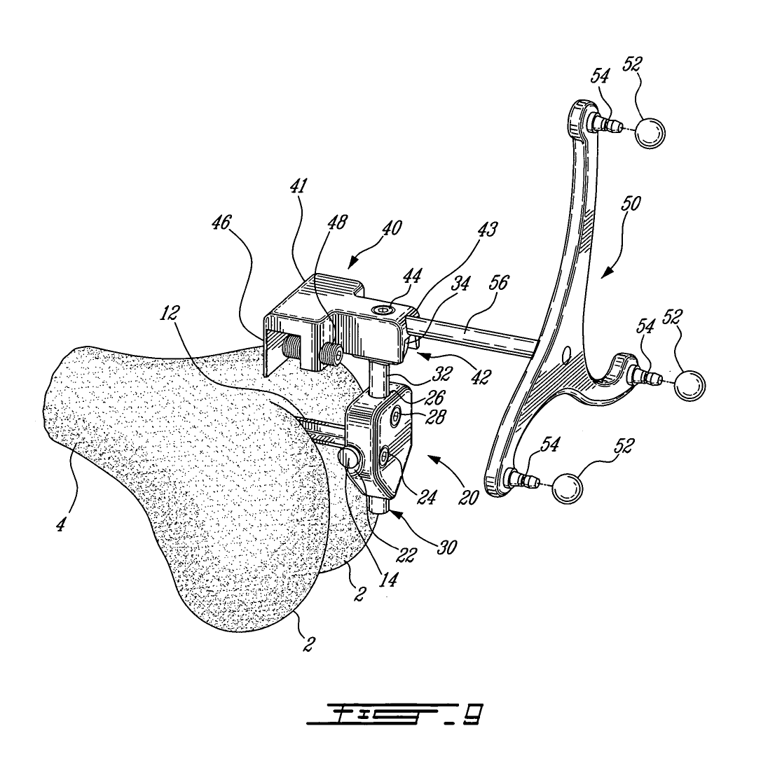

Referring to FIG. 9, the guide block holder 40, having opposed proximal 41 and distal 43 ends, is engaged to the antero-posterior slider 30 by sliding the guide block holder 40 onto the antero-posterior slider 30 such that the head 34 of the antero-posterior slider 30 slides within a corresponding shaped and configured aperture 42 in the guide block holder 40. Thus positioned, the guide block holder 40 may be translated generally in the direction of the proximal-distal axis, without being interfered with by the distal end of the femur 4, and rotated in the medio-lateral rotation plane. A proximal-distal translation/medio-lateral rotation friction locking screw 44 engages the head 34 of the antero-posterior slider 30 to retain a selected position relative to the antero-posterior slider 30. In an alternative embodiment (not shown), in a similar fashion as for the previous friction locking screws 24 and 28, friction locking screw 44 may be replaced by an endless screw, which engages an antero-posterior slider head having a series of indentations which are reciprocal to the threads of the proximal-distal translation/medio-lateral rotation endless screw. Referring back to FIG. 6, it is to be understood that if this alternative embodiment of the antero-posterior slider 30 is used, the guide block holder 40 may not be rotated in the medio-lateral rotation plane, thus providing one less degree-of-freedom.

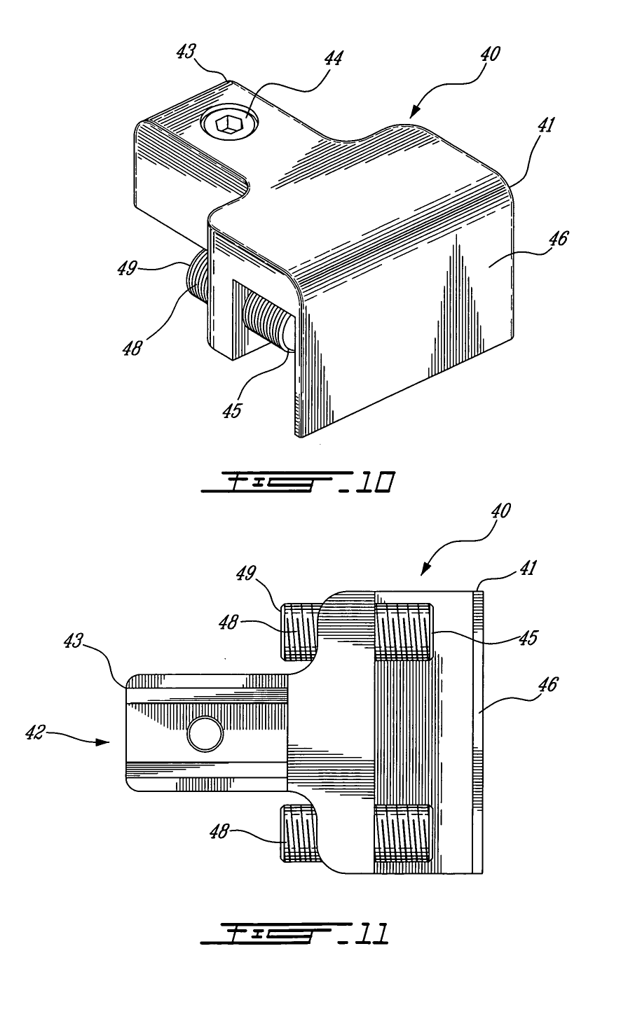

As shown in FIGS. 1 and 2, a conventional surgical tool guide block 70 having, for example, a number of drill guide holes 74 and a cutting guide slot 72, may be releasably engaged directly to universal positioning block assembly 100, via the guide block holder 40, which has a connector in the form of lip component 46 at its proximal end 41, as best seen in FIGS. 9, 10 and 11, that is sized and configured so that it may be inserted into a cutting guide slot 72 of the surgical tool guide block 70 or alternatively into another recess. One or more cutting guide locking screws 48, having a first 45 and second end 49, are provided to engage the surgical tool guide block 70, such that it may be held firmly in between the lip component 46 and the first end 45 of the cutting guide locking screws 48. The lip component 46 is preferably of a thickness such that it may be inserted, for example, in a snugly fitting relationship into any type of surgical tool guide block 70 cutting guide slot 72, the cutting guide locking screws 48 being operative in securing the surgical tool guide block 70 to the guide block holder 40 regardless of the difference between the thickness of the lip component 46 and the width of the cutting guide slot 72.

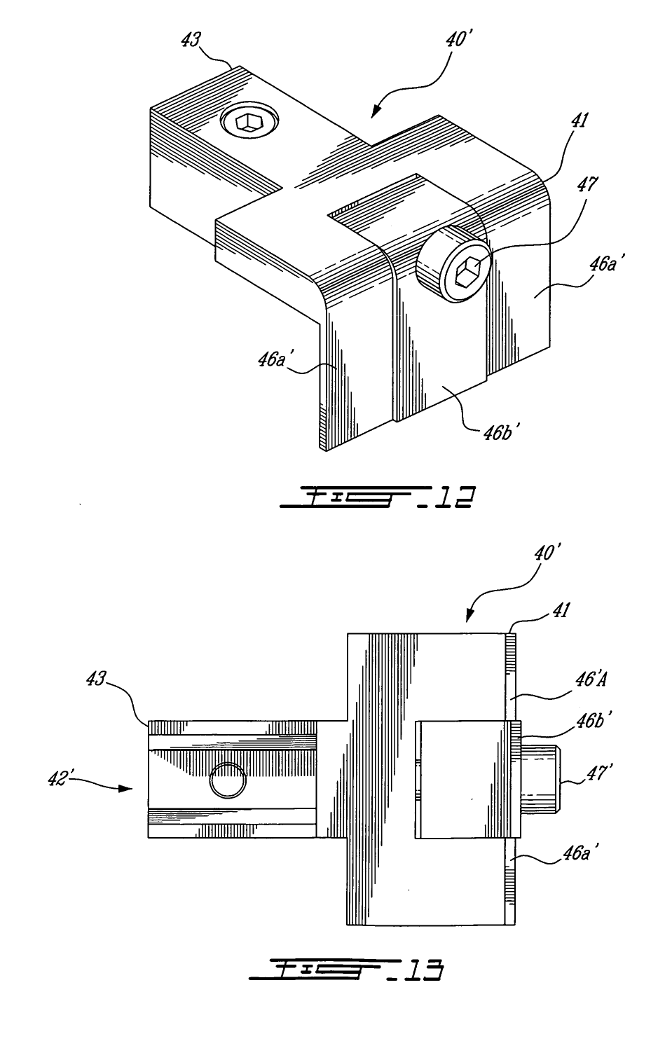

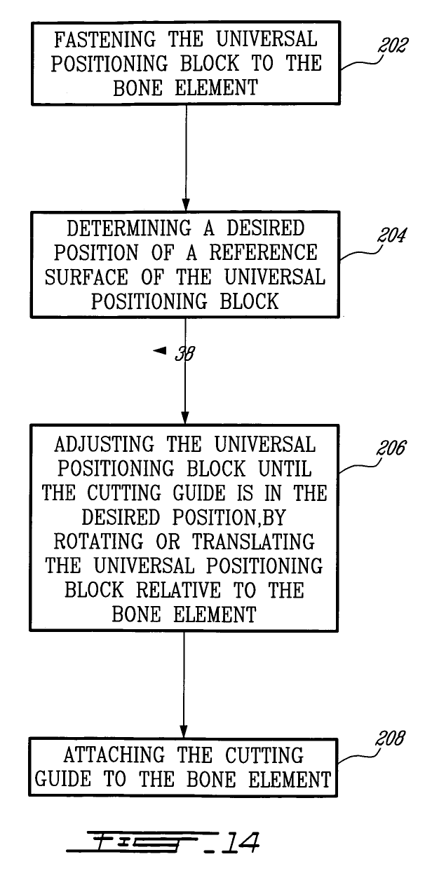

In an alternate embodiment, illustrated in FIGS. 12 and 13, the cutting guide holder lip component 46′ is actuated, having a fixed first connector part 46a′ and a displaceable second connector part 46b′, which may be displaced by rotating adjustment screw 47′. By rotating the adjustment screw 47′, the displaceable second connector part 46b′ may be displaced so as to become either in or out of horizontal alignment with the fixed first connector part 46a′. Thus, to secure the surgical tool guide block 70 to the guide block holder 40, the horizontally aligned fixed first connector part 46a′ and displaceable second connector part 46b′ are inserted into the cutting guide slot 72 and the adjustment screw 47′ rotated so that the fixed first connector part 46a′ and the displaceable second connector part 46b′ are horizontally displaced with respect to one another and are each biased against an opposite interior wall of the cutting guide slot 72.

Referring back to FIG. 9, the tracker member 50 comprises at least three detectable elements 52, engaged to the tracker member 50 via mounting posts 54. The detectable elements 52 may be, for example, spherical passive markers locatable by a camera-based, optical tracking CAS system. However, it is to be understood that active optical markers may equivalently be used as the detectable elements and that CAS systems using any other type of tracking elements, such as for example electromagnetically and acoustically detectable elements, may also similarly be employed. The tracker member 50 is connected, via the tracker stem 56, to distal end 43 of the guide block holder 40 although it may be affixed elsewhere to guide block holder 40. Thus, the guide block holder 40 being operatively engaged to the surgical tool guide block 70, as shown in FIG. 1, the tracker member 50 tracks the precise position of the cutting plane of the surgical tool guide block 70 by tracking the position of the lip component 46.

Referring back to FIGS. 1 and 2, showing the universal positioning block assembly 100 mounted to the distal end of a femur 4 by the bone anchor 10, and to FIG. 14 showing method steps involved with installing the positioning block assembly 100 and positioning the surgical tool guide block 70 on a femur 4. The degree of mobility of the positioning block assembly 100 permits significant simplification of the surgical procedures employed in certain surgeries, such as total knee replacement surgery. The sequence of steps composing the method involved with installing the positioning block assembly 100 and positioning the surgical tool guide block 70 on a femur 4 is described in the sequence of blocks 202 to 208. Generally, the first step, at block 202, comprises fastening the positioning block assembly 100 to the femur 4. As shown in FIG. 3, this is preferably done using the bone anchor 10, which is first aligned with the entrance point of the mechanical axis at the distal end of the femur 4 and introduced therein. Then, the main block 20, as best seen in FIG. 5, is slid unto the head 14 of the bone anchor 10 via main block aperture 22. Following which, the antero-posterior slider 30, as best seen in FIG. 7, is slid into main block aperture 26. Finally, as best seen in FIG. 1, the guide block holder 40, with a surgical tool guide block 70 engaged thereto and with the tracker member 50 connected to it, is engaged to the antero-posterior slider 30 by sliding the guide block holder 40 onto the antero-posterior slider 30 such that the head 34 of the antero-posterior slider 30 slides within the corresponding aperture 42 in the guide block holder 40.

The next step, at block 204, consist in determining a desired position of the surgical tool guide block 70, either by the CAS system itself, by the surgeon using the CAS system as a guide or independently by the surgeon, in order to determine what final position the universal positioning block assembly 100 should be moved into. It is to be understood that if the universal positioning block assembly 100 is to be used with a CAS system, a tracker element has to have been previously attached to the femur 4 so that the CAS system may determine the position of the universal positioning block assembly 100 relative to the femur 4. This final positioning of the positioning block assembly 100 has for effect the positioning of the surgical tool guide block 70 such that a drilled hole or a saw cut may be made in the femur 4 at a predetermined location that is required for the installation of an implant.

The step described at block 206 comprises adjusting the position and orientation of the universal positioning block assembly 100 until the surgical tool guide block 70 is located in the desired position that was previously determined at block 204. This may involve rotatably adjusting and translating the positioning block assembly 100 relative to the femur 4, using the CAS system, for example through a display generated by the CAS system, to aid in the correct orientation in each rotational axis of rotation and translation axis. Up to three rotational and three translational degrees of freedom are thereby possible, and the entire positioning block assembly 100, and thus surgical tool guide block 70, may be oriented in a desired plane, for example parallel to the distal cut to be made in the femur 4. The three possible rotations are in the flexion/extension plane, having for center of rotation the head 14 of the bone anchor 10, in the varus-valgus plane, having for center of rotation the body 32 of the antero-posterior slider 30 and in the medio-lateral plane, having for center of rotation the head 34 of the antero-posterior slider 30. As for the three possible translations, they are in the medio-lateral axis, along the head 14 of the bone anchor 10, the anterior-posterior axis, along the body 32 of the antero-posterior slider 30, and the proximal-distal axis, along the head 34 of the antero-posterior slider 30. As seen previously, the surgical tool guide block 70 is engaged to the guide block holder 40 which in turn is engaged to the tracker member 50 via the mounting posts 56. Thus, the position of the tracker member 50 is fixed relative to the surgical tool guide block 70, which permits the exact placement of the surgical tool guide block 70 by rotatably adjusting and translating the positioning block assembly 100 relative to the femur 4 using the CAS system.

Once the desired position and orientation of the universal positioning block assembly 100, and consequently of the positioning of the surgical tool guide block 70, is achieved, the step of block 208 is performed. This step comprises attaching the surgical tool guide block 70 to the femur 4 by drilling pin holes into the femur 4 using the necessary guide holes 74 in the surgical tool guide block 70, best seen in FIG. 2, and then inserting pins through the guide holes 74 and into the femur 4, securing the surgical tool guide block 70 to the femur 4. The entire positioning block assembly 100 may then be removed, and the femur 4 cut may be made to resect the chosen amount from the distal end of the femur 4.

The six degree-of-freedom adjustment that is possible by the universal positioning block assembly 100, as well as its guide block holder 40 design, permits it to be universally used in total knee replacement surgery, regardless of the type of implant line being used and of the surgical steps to be performed.

Although the universal positioning block assembly 100 has been described above with emphasis on the preparation of a femur for receiving the femoral portion of a knee replacement implant, the positioning block assembly 100 may also be used for the preparation of the tibia for the corresponding tibial portion of a knee replacement implant. The steps required to prepare the tibia, include: defining the tibial mechanical axis; using the positioning block assembly 100 to determine a desired rotational and translational alignment of a tibial proximal cutting guide and fastening it in place to the anterior surface of the proximal end of the tibia using the bone anchor 10; adjusting the universal positioning assembly 100 to ensure a desired posterior slop and level of tibial resection; inserting pins through the guide holes of the tibial cutting guide; removing the positioning assembly 100 and resecting the chosen amount of tibial bone. The positioning assembly 100 may further still be used for total replacement surgery of joints other than the knee, for example elbow replacement surgery or other related bone surgeries such as for example, unicondylar osteotomy, unicompartmental joint replacement, etc.

Although the present invention has been described by way of particular embodiments and examples thereof, it should be noted that it will be apparent to persons skilled in the art that modifications may be applied to the present particular embodiment without departing from the scope of the present invention.

What is claimed is:

1. A positioning block assembly (100) for use in bone surgery, to be releasably affixed to a bone element (4) to undergo surgery, said positioning block assembly (100) comprising:
a main block element (20) being releasably attachable to said bone element (4) such that said main block element (20) is rotatable in a flexion-extension rotation plane and translatable along a medio-lateral axis relative to said bone element (4);
a slider element (30) being slidably engaged with said main block element (20) such that it is translatable along an antero-posterior axis and rotatable in a varus-valgus rotation plane relative to said main block element (20); and
a holder element (40) being slidably engaged with said slider element (30) such that it is translatable relative to said slider element (30) along a proximal-distal axis, said holder element (40) being configured and disposed to releasably engage a surgical tool guide element (70).

2. The apparatus as defined in claim 1, wherein said holder element (40) is further rotatable in a medio-lateral rotation plane.

3. The apparatus as defined in claim 2 wherein said holder element (40) comprises an adjustment mechanism (44) for adjusting the rotation of said holder element (40) in said medio-lateral rotation plane and the translation of said holder element (40) in said proximal-distal axis.

4. The apparatus as defined in claim 3, wherein said adjustment mechanism (44) is selected from a group comprising an endless screw and a screw.

5. The apparatus as defined in claim 4, wherein said endless screw is operationally engaged to said slider element (30) providing substantially isolated adjustment in said medio-lateral rotation plane.

6. The apparatus as defined in claim 1, further comprising a bone anchoring element (10) having opposed first (11) and second (13) extremities, said first extremity (11) being releasably anchorable into said bone element (4) and said second extremity (13) being configured and disposed to be slidably and rotationaly engaged with said main block element (20).

7. The apparatus as defined in claim 6, wherein said second extremity (13) has a cylindrical cross section.

8. The apparatus as defined in claim 1, wherein said main block element (20) comprises a first adjustment mechanism (24) for adjusting the rotation of said main block element (20) in said flexion-extension plane and the translation of said main block element (20) in said medial-lateral axis.

9. The apparatus as defined in claim 8 wherein said main block element (20) further comprises a second adjustment mechanism (28) for adjusting the rotation of said slider element (30) in said varus-valgus rotation plane and the translation of said slider element (30) in said anterior-posterior axis.

10. The apparatus as defined in claim 9 wherein said holder element (40) comprises a third adjustment mechanism (44) for adjusting the translation of said holder element (40) in said proximal-distal axis.

11. The apparatus as defined in claim 8, wherein said first adjustment mechanism (24) is selected from a group comprising an endless screw and a screw.

12. The apparatus as defined in claim 9 wherein said second adjustment mechanism (28) is selected from a group comprising an endless screw and a screw.

13. The apparatus as defined in claim 10 wherein said third adjustment mechanism (44) is a screw.

14. The apparatus as defined in claim 11, wherein said endless screw is operationally engaged to said bone anchoring element (10) providing substantially isolated adjustment in said flexion-extension rotation plane.

15. The apparatus as defined in claim 12, wherein said endless screw is operationally engaged to said slider element (30) providing substantially isolated adjustment in said varus-valgus rotation plane.

16. The apparatus as defined in claim 1, wherein said holder element (40) comprises opposed proximal (41) and distal (43) ends, said proximal end (41) comprising a lip component (46) configured and disposed to be releasably engaged to said surgical tool guide element (70).

17. The apparatus as defined in claim 16, wherein holder element (40) further comprises fixation means for releasably engaging said lip component (46) to said surgical tool guide element (70).

18. The apparatus as defined in claim 17 wherein fixation means comprises at least one screw (48) having oppose first (45) and second (49) ends, said screw (48) being capable of displacement so as to releasably impinge said surgical tool guide element (70) between said first end (45) and said lip component (46).

19. The apparatus as defined in claim 18 wherein said surgical tool guide element (70) comprises an aperture (72) configured and disposed to receive therein said lip component (46).

20. The apparatus as defined in claim 16, wherein said lip component (46) comprises a fixed first connector part (46a′) and a displaceable second connector part (46b′), said second connector part (46b′) being displaceable between
a first position such that said second connector part (46b′) and said first connector part (46a′) are in horizontal alignment, and
a second position such that said second connector part (46b′) is horizontally displaced from said first connector part (46a′)
wherein said first connector part (46a′) and said second connector part (46b′) are both sized and configured to be inserted into said aperture (72) such that when said second connector part (46b′) is in said second position, said surgical tool guide element (70) is releasably engaged to said holder element (40) and when said second connector part (46b′) is in said first position, said surgical tool guide element (70) is disengaged from said holder element (40).

21. The apparatus as defined in claim 20 wherein said holder element (40) further comprises a screw (47′) operationally connecting said second connector part (46b′) to said holder element (40) such that rotation of said screw (47′) in a first direction displaces said second connector part (46b′) to said second position and rotating said screw (47′) in a second direction displaces said second connector part (46b′) to said first position.

22. The apparatus as defined in claim 1, wherein a tracker member (50) is connected to said holder element (40), said tracker member (50) comprises at least three detectable elements (52) configured and disposed so as to be tracked in three dimensional space by a computer assisted surgical system, thereby defining the location of said surgical tool guide element (70) releasably engaged to said holder element (40).

23. The apparatus as defined in claim 1, wherein said surgical tool guide element (70) is a surgical tool guide block.

24. The apparatus as defined in claim 23, wherein said surgical tool guide block comprises at least one of a group comprising a drill guide hole, a cutting guide slot, a rasp guide slot and a saw guide slot.

25. The apparatus as defined in claim 23, wherein said surgical tool guide block is a conventionally employed instrument used in non-computer assisted total joint replacement surgery.

26. The apparatus as defined in claim 1, wherein said bone surgery is joint replacement surgery.

27. The apparatus as defined in claim 26, wherein said joint is a knee.

28. A computer assisted bone surgery system comprising:
a positioning block assembly (100) to be releasably affixed to a bone element (4);
means for determining the position and orientation of said positioning block (100) relative to said bone element (4);
a surgical tool guide element (70) being releasably engaged to said positioning block assembly (100);
means for identifying a desired position of said positioning block assembly (100) relative to said bone element (4), such that said surgical tool guide element (70) is located in a selected position relative to said bone element (4), such that a cut can be made in said bone element (4) at said selected position; and
a display capable of indicating when said desired position of said positioning block assembly (100) is reached.

29. The apparatus as defined in claim 28, wherein said means for determining the position and orientation of said positioning block (100) is selected from a group comprising optical markers, electromagnetic markers and acoustic markers and further comprises a computer assisted surgery system.

30. The apparatus as defined in claim 28, wherein said means for identifying a desired position of said positioning block assembly (100) is a computer assisted surgery system.

31. The system as defined in claim 28, wherein said surgical tool guide element (70) is a surgical tool guide block.

32. The system as defined in claim 31, wherein said surgical tool guide block comprises at least one of a drill guide hole, a cutting guide slot, a rasp guide slot and a sawguide slot.

33. The system as defined in claim 29, wherein said surgical tool guide block is a conventionally employed instrument used in non-computer assisted total joint replacement surgery.

34. The system as defined in claim 28, further comprising means for determining and indicating where to fasten said positioning block assembly (100) on said bone element (4) such that said positioning block assembly (100) is located in said desired position.

35. The system as defined in claim 34, wherein said means for determining and indicating where to fasten said positioning block assembly (100) is a computer assisted surgery system.

36. The system as defined in claim 28, further comprising a bone anchoring element (10) having opposed first (11) and second (13) extremities, said first extremity (11) being releasably anchorable into said bone element (4) and said second extremity (13) being configured and disposed to be slidably and rotationaly engaged with said positioning block assembly (100), such that said positioning block assembly (100) may be selectively adjusted into said desired position.

37. The system as defined in claim 36, wherein said positioning block (100) comprises a tracker member (50) thereon, said tracker member (50) having at least three detectable elements (52) that may be located and tracked in three dimensional space by said computer assisted surgical system, thereby defining the location of said surgical tool guide element (70) releasably engaged to said positioning block assembly (100).

38. The system as defined in claim 28, wherein said bone surgery is joint replacement surgery.

39. The system as defined in claim 38, wherein said joint is a knee.

40. A method of installing a surgical tool guide element (70) on a bone element (4), said surgical tool guide element (70) being releasably engaged to a positioning block assembly (100), said method comprising:
fastening said positioning block assembly (100) to said bone element (4);
determining a desired position of said surgical tool guide element (70) engaged to said positioning block assembly (100) relative to said bone element (4);
adjusting at least one of the position and orientation of said positioning block assembly (100) until said surgical tool guide element (70) is in said desired position; and
securing said surgical tool guide element (70) on said bone element (4) at said desired position.

41. The method as defined in claim 40, wherein said surgical tool guide element (70) is surgical tool guide block.

42. The method as defined in claim 41, wherein said surgical tool guide block comprises at least one of a drill guide hole, a cutting guide slot, a rasp guide slot and a saw guide slot.

43. The method as defined in claim 41, wherein said surgical tool guide block is a conventionally employed instrument used in non-computer assisted total joint replacement surgery.

44. The method as defined in claim 40, further comprising using a computer assisted surgical system, communicable with said positioning block assembly (100), to determine and display position and orientation of said positioning block assembly (100) in relation to said bone element (4).

45. The method as defined in claim 44, wherein said computer assisted surgical system is used to determine said desired position of said surgical tool guide element (70).

46. The method as defined in claim 44, wherein said computer assisted surgical system is used to adjust said positioning block (100) such that said surgical tool guide element (70) is in said desired position.

47. The method as defined in claim 44, wherein said computer assisted surgical system is used to fasten said positioning block assembly (100) to said bone element (4) in a predetermined position.

48. The method as defined in claim 47, wherein said computer assisted surgical system is used to adjust at least one of the position and orientation of said positioning block assembly (100) while fastening said positioning block assembly (100) such that said positioning block assembly (100) is in said predetermined position.

49. The method as defined in claim 40, further comprising using said positioning block assembly (100) for joint replacement surgery.

50. The apparatus as defined in claim 49, wherein said joint is a knee.

51. The method as defined in claim 40, further comprising using a bone anchoring element (10) to fasten said positioning block assembly (100) to said bone element (4), such that said positioning block assembly (100) may selectively be rotatably and translatively orientated relative to said bone element (4).
